(19) United States
(12) Patent Application Publication
(10) Pub. No.: US 2021/0000643 A1
(43) Pub. Date: Jan. 7, 2021

(54) DEVICE FOR CARRYING OUT A SHIRODHARA TREATMENT AND METHOD FOR ESTABLISHING A LIQUID FLOW WHICH FLOWS OUT OF AN OUTLET

(71) Applicant: Ayavaya BV, Gravenhage (NL)

(72) Inventors: ILAN Karavani, Jerusalem (IL); Merijn Sanders, Berlin (DE)

(73) Assignee: Ayavaya BV, Gravenhage (NL)

(21) Appl. No.: 16/979,258

(22) PCT Filed: Mar. 12, 2019
(86) PCT No.: PCT/EP2019/056122

§ 371 Date: Sep. 9, 2020

(30) Foreign Application Priority Data
Mar. 19, 2018 (BE) 2018/5180

(51) Int. Cl. A61F 7/00

(52) U.S. Cl. CPC A61F 7/0085; A61F 7/007; A61F 2007/0007; A61F 2007/0059; A61F 2007/0086; A61F 2007/0069; A61F 2007/0096

(57) ABSTRACT

Device for carrying out a Shirodhara treatment in which a liquid is poured over the forehead of a person, whereby said device comprises an outlet for the liquid and a pump and a supply line running from said pump to said outlet, whereby said outlet is can move reciprocally in a regular manner, whereby said device is provided with a heating unit for heating liquid to a target temperature, whereby said heating unit comprises a heating element, whereby said heating element is disposed in or around said supply line, whereby said heating unit comprises a temperature sensor for measuring the temperature of liquid, whereby said device comprises a control unit to regulate the power of said heating element on the basis of the temperature measured by said temperature sensor.

### CROSS REFERENCE TO RELATED APPLICATIONS

This non-provisional utility patent application claims the benefit of and priority to PCT Application Serial No. PCT/EP2019/056122, filed Mar. 12, 2019, entitled “Device for Carrying Out a Shirodhara Treatment and Method for Establishing a Liquid Flow which Flows Out of an Outlet, which claims benefit of and priority to Belgium Patent Application Serial No. 2018/5180, Filed Mar. 19, 2018, the entire contents of both applications are hereby incorporated herein by reference.

### BACKGROUND

The present invention relates to a device for carrying out a Shirodhara treatment and a method for establishing a liquid flow which flows out of an outlet, in particular for using this in a Shirodhara treatment.

A Shirodhara treatment is a traditional Indian treatment for preventing or treating certain disorders and for relaxation. In such a treatment, a trickle of warm liquid, for example oil, is poured over the forehead of a person to be treated during a certain period with a reciprocal movement.

For an optimal effect of this treatment, the liquid must have a temperature which is as constant as possible and also a rate of flow which is as constant as possible, and must certainly not start dripping. In order to achieve this, traditional therapists must follow a lengthy training.

Because of this, it is not easy in practice to apply such a treatment widely: the number of proficient therapists is limited and it is very labour-intensive.

Devices are known for carrying out a Shirodhara treatment partially or fully automated, such as from DE102007043558A1 and EP1039871B1. These are not or only partly successful, however, in maintaining a sufficiently constant temperature and rate of flow of the flowing liquid.

### SUMMARY

The present invention seeks to provide a solution for the above-mentioned and other disadvantages and thereto provides a device for carrying out a Shirodhara treatment in which a liquid is poured over the forehead of a person, whereby said device comprises an outlet for the liquid, a pump for the liquid and a supply line for the liquid, whereby said supply line runs from the pump to the outlet, whereby said device is provided with a heating unit for heating liquid flowing through said supply line to a target temperature.

Said target temperature is obviously a target temperature downstream from said heating unit.

Thanks to the heating of the liquid flowing through said supply line, a high temperature accuracy and high heating speed of the liquid can be achieved, much better than in the case where a reservoir with liquid is heated via a thermostat.

Because of this, said device is also suitable for accommodating changing circumstances, such as a fluctuation in input temperature of the liquid, without this having much effect on the temperature of the liquid in the outlet.

As usual in a Shirodhara treatment, said outlet is arranged in this regard to move reciprocally in a regular manner, preferably at a frequency of not less than 2 and not more than 30 full reciprocal movements per minute.

In this regard, said outlet is arranged to move reciprocally in a regular manner over a distance that is not less than 2 cm and not more than 20 cm.

This is achieved by said device being provided with means for moving said outlet reciprocally in a regular manner over the said distance.

The outlet preferably has a diameter of not less than 2 mm and not more than 30 mm.

The liquid can here be any liquid common in Shirodhara treatments, such as vegetable oil, milk, vegetable juices or water.

In a preferred embodiment, said heating unit comprises a temperature sensor for measuring the temperature of the liquid and said heating unit comprises a heating element, whereby said device is provided with a control unit to regulate the power of the heating element on the basis of the temperature measured by said temperature sensor.

Preferably the heating element is an electrical heating element.

For clarity, it is remarked that by the power of the heating element is meant the electrical power which is supplied to the heating element and which is converted into thermal power by the heating element, not the maximum power for which the heating element is designed.

Normally speaking, the device is a stand-alone, movable device with limited dimensions. Preferably the largest dimension of said device is less than 5 m, with greater preference less than 4 metres and with still greater preference less than 3 metres. This obviously also implies that the distance between the pump and said outlet is preferably smaller than the said dimensions.

In a further preferred embodiment, said temperature sensor is disposed upstream from the heating element, whereby said control unit is arranged to regulate the power of the heating element on the basis of a target temperature of the liquid downstream from the heating element and the temperature measured by said temperature sensor of the liquid.

Preferably this happens in addition on the basis of a set, measured or calculated rate of flow of the liquid and on the basis of a measured or set value of the heat capacity of the liquid.

In this way, by means of forward coupling, a predictive calculation is made of the required power to reach the desired target temperature.

Since there is always a certain delay in a heating system, a predictive calculation leads to less fluctuation of the ultimately reached result, certainly in the case that the temperature of the liquid to be heated before the heating element is not constant.

Because of this, a maximum fluctuation of the temperature at said outlet can be achieved which is smaller than 0.5° C.

Nevertheless, a temperature regulation by feedback is also possible, so that in a preferred embodiment said temperature sensor is disposed downstream from the heating element, whereby said control unit is arranged to regulate the power of the heating element on the basis of the difference between a target temperature of the liquid downstream from the heating element and the temperature measured by the temperature sensor.

In cases in which there is little fluctuation in the temperature of the liquid to be heated, this leads to a more accurate end result.

In another preferred embodiment, said heating unit comprises two or more combinations of a heating element and a temperature sensor, whereby the combinations, seen in the direction of flow of the liquid, are disposed behind each other, and whereby said device is arranged to regulate the temperature of the liquid downstream from each combination to a separate target temperature.

This can alternatively be described as: said heating unit is provided with two or more separate heating steps, whereby each of the heating steps has a separate control with a temperature sensor to regulate the liquid temperature to a target temperature immediately downstream from the heating step.

In this way, part of the required heating can be done by each combination. Hereby the deviation of the target temperature per combination, that is, per heating step, is smaller, so that the fluctuation in the target temperature after the full heating unit is also smaller.

Preferably, said device is provided with three or four of said combinations. In practice, this proves to provide a good compromise between limiting the temperature fluctuation and the complexity of said device.

In another preferred embodiment, said heating unit comprises a said heating element further comprising a temperature sensor which is disposed upstream from said heating element and a temperature sensor which is disposed downstream from said heating element.

This allows a self-learning control to be applied.

In another preferred embodiment, said device is provided with a return line to return liquid which has been poured over the forehead of a person to the pump or to said supply reservoir for the pump. Hereby a closed liquid circuit can be formed so that the liquid can be re-used.

In another preferred embodiment, said device is provided with a by-pass line which runs from a position downstream from said heating unit but upstream from said outlet to the pump or to said supply reservoir for the pump, whereby said device is provided with means to convey liquid alternatively to said outlet or to the by-pass line.

This allows the liquid already to be made to flow and to be brought to the desired temperature before the person to be treated takes their place, and also prevents the person to be treated being disturbed by liquid flowing out of said outlet upon taking their place and upon preparation for the treatment.

In another preferred embodiment, said device is provided with a supply reservoir for liquid for the pump, whereby said supply reservoir is preferably removable and able to be reinstalled.

In this regard, the pump is arranged to draw the liquid out of the supply reservoir and to pump it to said outlet.

This enables an easy replacement of the liquid, so that successive persons to be treated can easily be treated with different liquids, or could even use a personal quantity of liquid, which they could prefer for reasons of hygiene.

In another preferred embodiment, said device comprises a flow gauge for measuring the rate of flow of the liquid through said supply line, and means to adapt the operational state of the pump on the basis of the measured rate of flow in order thereby to regulate the rate of flow to a target value.

Hereby a very constant liquid flow is ensured, which on the one hand is in itself important for an optimal Shirodhara treatment, and on the other hand decreases the temperature fluctuations of the liquid.

The invention further comprises a method for establishing a liquid flow which flows out of an outlet, wherein said outlet moves reciprocally in a regular manner at a frequency lying between twice per minute and thirty times per minute, wherein liquid is pumped out of a reservoir and flows through a line to the outlet, wherein the liquid flow has a rate of flow of not less than 100 ml per minute, and preferably not less than 200 ml per minute and not more than 3000 ml per minute, and preferably not more than 2000 ml per minute, wherein the liquid is heated by a heating element disposed in or around said line, wherein the liquid is heated to a target temperature by the heating element.

This is an easy and reliable method for establishing a liquid flow which is suitable for a Shirodhara treatment.

In a preferred variant, the heating element is an electrical heating element, wherein a temperature sensor measures the temperature of liquid flowing through said line, wherein a control unit regulates the electrical power of the heating element on the basis of the temperature measured by temperature sensor.

In this method a device according to the invention is preferably used.

In a preferred variant, the temperature of the liquid is regulated by said control unit such that it has a maximum temporary deviation of 0.5° C. compared with the time-averaged temperature of the liquid.

In a preferred variant, the liquid is heated to a temperature lying between 30° C. and 50° C., and preferably between 35° C. and 45° C.

In a further preferred variant, the rate of flow is regulated such that it has a maximum temporary deviation of 20%, and preferably of 10%, compared with the time-averaged rate of flow.

The invention also comprises a method for carrying out a Shirodhara treatment to allow a person to be relaxed or to increase their general well-being, thus not aimed at the treatment or prevention of a physical or mental disorder, wherein a liquid flow is poured over the forehead of the person, wherein the liquid flow is established by means of an aforementioned method according to the invention.

In this regard, the distance between the forehead of the person and said outlet is preferably not less than 2 mm and not more than 400 mm.

The invention further comprises a method for heating a liquid which, after heating, is used for a Shirodhara treatment, wherein the liquid flows through a line and along a heating element disposed in or around said line, wherein the liquid is heated by the heating element to a target temperature, and wherein the liquid which flows through said line preferably flows along a first such heating element and subsequently flows along a second such heating element, wherein the liquid is heated by the first heating element to a first target temperature and is heated by the second heating element to a second target temperature, wherein the second target temperature is higher than the first target temperature.

### BRIEF DESCRIPTION OF THE DRAWINGS

In order to clarify the invention, a preferred embodiment of a device according to the invention is described below, with reference to the following figures, whereby

FIG. 1 schematically shows a device according to the invention; and

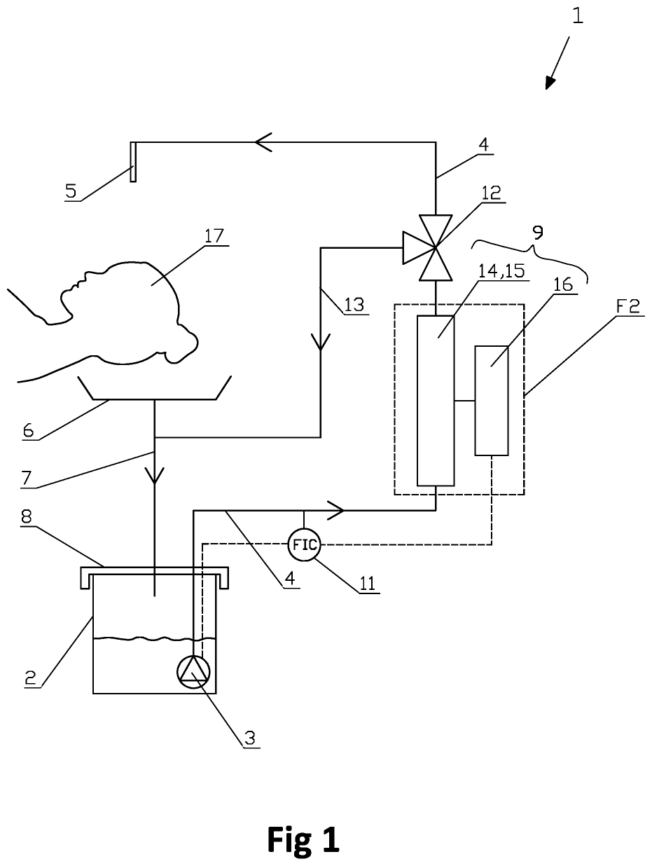

FIG. 2 schematically shows the part of said device designated by F2 in more detail.

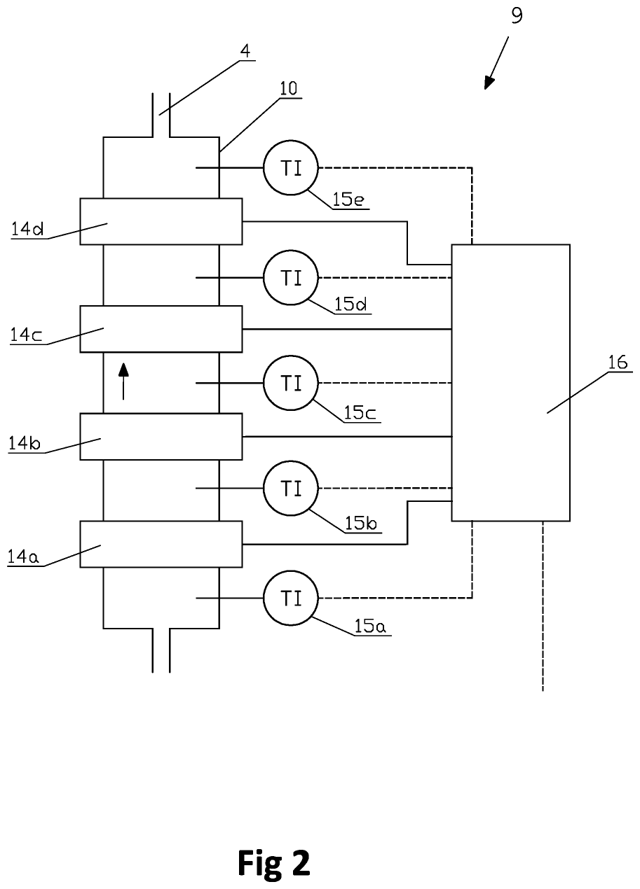

### DETAILED DESCRIPTION

Said device 1 shown in the figures comprises a reservoir 2 of approximately two litres, filled in this example with sesame oil.

In this reservoir 2, a submersible pump 3 is disposed. A supply line 4 for the sesame oil runs from the submersible pump 3 to an outlet 5. A collector receptacle 6 is disposed under said outlet 5 to collect liquid flowing out of said outlet 5 and to return it via a return line 7 to said reservoir 2. An open space is provided between said outlet 5 and the collector receptacle 6 to accomodate the head of a person to be treated.

Said reservoir 2 is provided with a cover 8, and can be removed while said cover 8 stays connected to the rest of said device 1.

Said outlet 5 has a diameter of 6 mm and is arranged by means of a motorised drive, not shown, to move reciprocally over a distance of approximately 10 cm at a frequency of seven times per minute.

A heating unit 9 is disposed around said supply line 4. Said supply line 4 is made of plastic and has a diameter of 10 mm. At said heating unit 9, said supply line 4 has a widened part 10 with a diameter of 40 mm. This widened part 10 is made of copper.

Because said supply line 4, except for the widened part 10, is implemented in plastic, heat loss is avoided. Additionally, said supply line can be thermally insulated.

A flow gauge 11 is disposed between the submersible pump 3 and said heating unit 9 with a control to drive said submersible pump 3 so that a constant rate of flow of 750 ml sesame oil per minute is realised.

A three-way valve 12 is disposed between said heating unit 9 and said outlet 5, to which a by-pass line 13 is coupled which runs directly to said return line 7.

Said return line 7 and the by-pass line 13 are also made of plastic and also have a diameter of 10 mm.

The heating unit comprises four annular electrical heating elements 14 which are disposed behind each other around the widened part 10 of said supply line 4, further to be mentioned, in the order of the direction of flow of the sesame oil, the first heating element 14a, the second heating element 14b, the third heating element 14c and the fourth heating element 14d.

It is remarked that one or more heating element or elements is or are generally designated as reference 14, while for specific heating elements the references 14a, 14b, 14c and 14d are used.

The heating unit 9 further comprises five thermocouples 15 which are disposed in the widened part, namely a thermocouple upstream from the heating elements, further to be referred to as the first thermocouple 15a, three thermocouples which are disposed at positions between said heating elements 14, further to be referred to as the second thermocouple 15b, the third thermocouple 15c and the fourth thermocouple 15d, and a thermocouple downstream from the fourth heating element 14d, further to be referred to as the fifth thermocouple 15e.

It is remarked that one or more thermocouple or thermocouples are generally designated as reference 15, while for specific thermocouples the references 15a, 15b, 15c, 15d and 15e are used.

Said heating unit 9 further comprises a PLC 16, which is connected with the thermocouples 15 and with said heating elements 14 in such a manner that each heating element 14 can be independently driven by the PLC.

Said flow gauge 11 is data-transferably connected with the PLC 16.

The direction of flow of the sesame oil, in the case that said submersible pump 3 is in operation, is indicated in the figures by means of arrows.

The operation of said heating unit 9 is as follows:

A target temperature of the sesame oil after the fourth heating element 14d is entered in the PLC 16 by the user, in this example 40.0° C. The oil flow rate as set by means of said flow gauge 11, and the density and the heat capacity of the sesame oil, are also entered in the PLC 16.

Target temperatures of the sesame oil after the third heating element 14c, the second heating element 14b and the first heating element 14a respectively are now determined by said PLC 16, each time a fixed value, for example 1.0° C., lower than the target temperature after the following heating element 14.

The target temperature after the third heating element 14c is then, in this example, calculated at 39.0° C., after the second heating element 14b at 38.0° C. and after the first heating element 14a at 37.0° C.

Each heating element 14 is now independently driven by said PLC 16, on the basis of the temperature measured by said thermocouple 15 disposed immediately upstream from the related heating element 14.

In this regard, the electrical power to be supplied by said PLC 16 to the related heating element 14 is calculated as follows:

P=F*C*ρ*Q*(T2−T1)

where P is the electrical power, where F is an experimentally determined factor known to said PLC 16 which compensates for heat losses of said heating elements 14, where ρ is the density of the sesame oil, where Q is the volumetric flow rate of the sesame oil, where T2 is the target temperature immediately after the related heating element 14 and where T1 is the temperature measured by the related thermocouple 15 of the sesame oil immediately before the related heating element 14.

The electrical power to be supplied to said heating elements 14 is adapted by means of pulse width modulation, better known as PWM.

If the sesame oil, for example at the first thermocouple 15a, has a temperature of 30° C., said PLC 16 will calculate how much power is needed to heat the sesame oil by the first heating element 14a to the target temperature of 37° C. applicable downstream from the first heating element, and to supply this power to the first heating element 14a.

In this regard, a difference can of course occur between the temperature actually reached and the said 37° C. Considering the relatively large temperature difference of 7.0° C. between the temperature before and after the first heating element, this difference can be relatively large.

Since the following heating elements 14b, 14c, 14d each only need to realise a small additional temperature increase of 1.0° C., the differences between the actual oil temperature and the target temperatures at the third thermocouple 15c, fourth thermocouple 15d and fifth thermocouple 15e will become successively smaller, so that an extremely constant temperature will be obtained at the fifth thermocouple 15e.

Also, a disturbance of the inlet temperature, for example by adding oil having another temperature, can be well accommodated by said heating unit 9.

The temperature at the fifth thermocouple 15e is not used by said PLC 16 in the aforementioned embodiment to regulate the electrical power, but is measured in order, during start-up, to be able to determine whether the sesame oil is sufficiently heated to be able to start the treatment and to be able to detect an operational fault condition.

In a first alternative, heating unit 9 can use the rate of flow measured by said flow gauge 11 to perform a more accurate calculation.

In a second alternative, said PLC 16 can be made self-learning by comparing the temperature actually reached after a heating element 14 with the target temperature after this heating element 14. Hereby the oil flow rate, the density, the heat capacity and the factor F no longer need to be set, but the arithmetic product hereof can be determined by said PLC 16.

In this regard, for example, the temperature at the second thermocouple 15b is not only used to calculate the power to be supplied to the second heating element 14b, such as in the embodiment described above, but also to calculate a numerical value of the said arithmetic product of the oil rate, the density, the heat capacity and the factor F.

The remaining operation of said device 1 is as follows:

Upon commencement of the use of said device 1, said reservoir 2 is filled with sesame oil to be used, or an already filled reservoir 2 is placed under said cover 8. Said device 1 is subsequently turned on. In this regard, said three-way valve 12 is set such that the full oil flow runs via said by-pass line 13.

Said person to be treated 17 can now calmly take their place under said outlet 5. At the same time, the sesame oil can already be pre-heated. As soon as the sesame oil reaches the desired temperature of 40.0° C., the three-way valve 12 is switched, so that the full oil flow, via said outlet 5 and in a constant flow of 750 ml/minute at a constant temperature of 40° C., flows reciprocally over the forehead of the person to be treated 17 by the reciprocal movement of said outlet 5.

Via said collector receptacle 6 and said return line 7, the oil flows back to said reservoir 2 to then be pumped up again.

Hereby the oil obviously cools down somewhat. The oil is heated again to a temperature of 40° C. by said heating elements 14, however, so that a flow at a constant rate of flow and a constant temperature is poured out over the forehead of said person 17.

It is remarked that said return line 7 is optional, certainly if a cheap liquid is used.

1. A device for carrying out a Shirodhara treatment in which a liquid is poured over a forehead of a person, wherein said device comprises an outlet for the liquid and a pump and a supply line for the liquid, whereby said supply line runs from said pump to said outlet, whereby said outlet is arranged to move reciprocally, whereby said device is provided with a heating unit for heating the liquid flowing through said supply line to a target temperature, whereby said heating unit comprises a heating element, whereby said heating element is disposed in or around said supply line, whereby said heating unit comprises a temperature sensor for measuring a temperature of the liquid flowing through said supply line, whereby said device comprises a control unit to regulate an amount of electrical power which is applied to said heating element based on the temperature measured by said temperature sensor.

2. The device according to claim 1, wherein said outlet is arranged to move reciprocally at a frequency lying between two and thirty full reciprocal movements per minute.

3. The device according to claim 2, wherein said outlet is arranged to move reciprocally over a distance that is not less than two centimetres and not more than twenty centimetres.

4. The device according to claim 4, wherein said device is provided with a supply reservoir for said pump.

5. The device according to claim 4, wherein said device is provided with a return line to convey liquid which is poured out from said outlet back to said pump or to said supply reservoir.

6. The device according to claim 5, wherein the distance between said pump and said outlet is less than 5 metres.

7. The device according to claim 6, wherein the device is provided with a flow gauge to measure the rate of flow of the liquid through said supply line and is provided with means to adapt an operational state of said pump based on a measured rate of flow in order to thereby regulate the rate of flow of the liquid.

8. The device according to claim 7, wherein said heating element is an electrical heating element which is disposed around said supply line, whereby said temperature sensor is disposed upstream from said heating element, whereby said control unit is arranged to regulate the amount of electrical power which is supplied to said heating element on the basis of the target temperature of the liquid downstream from said heating element and the temperature measured by the temperature sensor and a set or measured liquid flow rate.

9. The device according to claim 8, wherein said device is provided with two or more combinations of said heating element and said temperature sensor, whereby the combinations, in regards to a direction of flow of the liquid, are disposed behind each other, and whereby said device is arranged to regulate the temperature of the liquid downstream from each heating element to a separate target temperature.

10. The device according to claim 9, wherein the device is provided with three or more of said combinations.

11. A method for establishing a liquid flow which flows out of an outlet, wherein said outlet moves reciprocally at a frequency lying between twice per minute and thirty times per minute, wherein a liquid is pumped out of a reservoir and flows through a line to said outlet, wherein a liquid flow has a rate of flow of not less than 100 ml per minute and not more than 3000 ml per minute, wherein the liquid is heated by a heating element disposed in or around said line, wherein the liquid is heated to a target temperature by said heating element.

12. The method according to claim 11, wherein said outlet moves reciprocally over a distance lying between two centimetres and twenty centimetres.

13. The method according to claim 12, wherein said heating element is an electrical heating element, wherein a temperature sensor measures a temperature of liquid flowing through said line, wherein a control unit regulates an amount of electrical power which is supplied to said heating element based on the temperature measured by the temperature sensor.

14. The method according to claim 13, wherein a device according to claim 10 is used to establish the liquid flow.

15. The method according to claim 14, wherein the method is utilized for carrying out a non-therapeutic Shirodhara treatment in which the liquid flow is established and poured over a forehead of a person.

16. The device according to claim 3, wherein said device being provided with means for moving said outlet reciprocally over the said distance.

17. The device according to claim 1, wherein said temperature sensor is disposed upstream from the heating element, whereby said control unit is arranged to regulate an amount of electrical power which is supplied to said heating element on the basis of a target temperature of the liquid downstream from the heating element and the temperature measured by said temperature sensor of the liquid.

18. The device according to claim 1, wherein said heating unit is provided with two or more separate heating stages, whereby each of the heating stages has a separate control with a temperature sensor to regulate the liquid temperature to a target temperature immediately downstream from the heating stage concerned.

19. The device according to claim 4, wherein said device is provided with a return line to convey liquid which is poured out from said outlet back to said supply reservoir.

20. A method for heating a liquid which, after heating, is used for a Shirodhara treatment, wherein the liquid flows through a line and along a heating element disposed in or around said line, wherein the liquid is heated by the heating element to a target temperature.
